(19) United States
(12) Patent Application Publication
(10) Pub. No.: US 2023/0150116 A1
(43) Pub. Date: May 18, 2023

(54) ORTHOPAEDIC DEVICE FOR SUPPORTING A LOWER EXTREMITY OF A USER

(71) Applicant: OTTOBOCK SE & CO. KGAA, Duderstadt (DE)

(72) Inventors: Torsten PARTH, Egelsbach (DE); Benjamin SCHIRRMEISTER, Göttingen (DE); Jonas BORNMANN, Duderstadt (DE); Paul HUNGER, Kirchgellersen (DE)

(21) Appl. No.: 18/156,609

(22) Filed: Jan. 19, 2023

Related U.S. Application Data
(63) Continuation-in-part of application No. PCT/EP2021/070145, filed on Jul. 19, 2021.

(30) Foreign Application Priority Data
Jul. 21, 2020 (DE) 10 2020 119 166.9

(51) Int. Cl. B25J 9/00; B25J 17/00

(52) U.S. Cl. CPC B25J 9/0006; B25J 17/00

(57) ABSTRACT

An orthopedic device for supporting a lower limb and/or a lower back of a user includes at least one upper joint element, at least one lower joint element which can be pivoted relative to the upper joint element, and at least one actuator configured to apply a torque to the upper joint element and/or the lower joint element. Application of a torque pivots the upper joint element and the lower joint element relative to each other. The orthopedic device also includes at least one ground contact element which is connected to the lower joint element such that when the orthopedicdevice is in the mounted state, the torque applied by the at least one passive actuator is transmitted by the ground contact element to a ground on which the user is located.

### CROSS-REFERENCE TO RELATED APPLICATIONS

This application is a continuation-in-part of international application PCT/EP2021/070154 filed Jul. 19, 2021, which claims priority to German Application 10 2020 119 166.9 filed Jul. 21, 2020.

### FIELD OF THE INVENTION

The invention relates to an orthopedic device for supporting a lower extremity and/or a lower back of a user, the orthopedic device comprising at least one upper joint element, at least one lower joint element that can be pivoted relative to the upper joint element, and at least one actuator that is configured to apply a torque to the upper joint element and/or the lower joint element by which the upper joint element can be pivoted relative to the lower joint element.

### BACKGROUND

Such devices have been known from the prior art for many years and are used especially during lifting to support the person who is to lift a heavy object, for example. In addition, such devices are used for persons who have to work in a bent position.

US 443 113, for example, describes a device that was designed for this purpose. It features upper leg elements that are arranged on the wearer’s upper leg. The device is also arranged on the wearer’s upper body via shoulder straps. Between the shoulder straps and upper leg elements there are leaf spring elements, which are bent when the wearer bends down, thereby charging them with potential energy. The leaf spring elements apply a force to the upper body that supports the extension of the body. However, it is disadvantageous that the resulting force is always exerted when an angle between the upper body and the upper leg changes. It is consequently exerted when climbing stairs or sitting, for example, which is at least uncomfortable, but also potentially both disruptive and uncomfortable.

Devices that are similar in principle are known from US 2017/0196712 A1 and US 2017/0360588 A1, for example. However, the force supporting the lower back or the upper body, which should make it easier for the person to straighten up, is not always exerted. In the first prior art named, it is not until a certain angle of inclination is exceeded, i.e. when the angle between the upper body element of the device and the upper leg element of the device falls short of a predetermined angle, that the force is exerted. Up to this angle, the upper body can be tilted relative to the upper leg without an actuator or energy store being charged with energy. Nevertheless, a supporting force always occurs in this device when the upper body is at an angle relative to the upper leg that is smaller than the predetermined limit angle, i.e. the upper body is tilted relative to the upper leg.

In the devices mentioned, the applied force is applied to the upper leg, so that it is loaded almost perpendicular to the longitudinal direction of the femur. In the event of prolonged use, which occurs particularly when the devices are used to relieve users in their work, this may cause problems and pain, as the upper leg is not designed for loads in this direction.

The prior art has therefore long sought to transfer loads to the ground rather than to the upper leg. US 9,808,073 B1, US 8,474, 672 B2 and US 9,492300 B2 disclose such devices, which aim to support the user when carrying a load. The load, which is always carried above the waist, for example in the form of a backpack or tools, is transferred to the ground via a frame in various configurations. This usually restricts the freedom of movement of the legs. Due to the path of the force flow, these devices from the prior art only work when the user is standing upright or walking. However, as soon as the user bends or leans forward, for example to lift a heavy object, these devices are not able to deliver the required and desired support.

US 9,980,873 B2 discloses a support device that features a knee joint, by means of which a first limb is connected to a second limb such that it can be pivoted. An actuator is arranged between the two limbs which, in a first operating mode of the device, applies a force to the two limbs when the knee is bent. In a second operating mode, on the other hand, no force is applied, even when the knee is bent. As a result, a person’s knee is supported, for example when they squat, while a bent knee in the swing phase of a step does not experience any supporting force. Therefore, some of the embodiments depicted feature a ground sensor which can detect whether the user’s leg is on the ground. Such a sensor can be arranged, for example, in a wearer’s shoe.

### SUMMARY

The invention is therefore based on the task of further developing a device in such a way that the force is also dissipated into the ground, in particular, when the user bends over or leans forward.

The invention solves the addressed task with an orthopedic device that comprises at least one ground contact element, which is connected to the lower joint element in such a way that, when the device is in the mounted state, as a result of the torque applied by the at least one passive actuator, a force is transmitted by means of the ground contact element to a ground on which the user is located.

In one embodiment the at least one actuator is a passive actuator.

Within the scope of the present invention, a passive actuator is understood to mean an actuator that works without a motor and external drive. Its energy, which it requires to exert the force, is generated by the movement of various components of the orthopedic device. Specifically, the at least one passive actuator is charged with energy when the upper joint element moves relative to the lower joint element. In particular, this movement refers to a pivoting, which preferably occurs when the user of the device leans forward or bends over. An orthopedic device is, in particular, an orthosis or an exoskeleton. Consequently, a passive actuator does not need any source of energy other than the wearer’s body or gravity.

The actuator preferably transmits its force to a force application lever which is connected to the respective component and can be pivoted with it about the pivot axis. As a result, the force exerted by the actuator becomes a torque. The effective length of the force application lever, in particular the force application point at which the actuator engages the force application lever, can be adjustable. Preferably, the force application point can be adjusted by means of a motor which, for example, displaces the force application point along the force application lever. Even if a motor is used or provided for this purpose, for the purposes of the present application it is referred to as a passive actuator, provided that the actuator itself is not motor-driven.

In another embodiment the actuator is an active actuator. Particularly preferably, the actuator includes a motor that can be actuated to apply the required torque.

For the present invention, an active actuator or a passive actuator may be advantageous. The passive actuator has the advantage of not requiring a separate power source or energy storage device, and thus is inexpensive, lightweight, and requires little installation space. An active actuator has the advantage that it is not necessary for the user of the device to charge with energy a passive actuator or energy storage device to obtain the supportive action of the actuator. The embodiment with active actuator is therefore particularly advantageous for physically weak or limited persons or in situations where a particularly large force and/or torque is required.

The passive actuator is preferably arranged in such a way that it applies a torque to the lower joint element and the upper joint element. Thanks to this torque, the two elements can be pivoted relative to one another.

Preferably, the torque applied by the at least one passive actuator acts in the direction of extension of the joint, which is composed of the upper joint element and the lower joint element. In this case, the direction of extension is the pivot direction that extends the joint, while the opposite direction, in which the joint is bent, is the so-called flexion direction.

The ground contact element is preferably connected to the lower joint element via at least one force transmission element. The force transmission element can be a splint or a rod, for example. Preferably, it is configured to transmit compressive forces. The force transmission element is preferably arranged at the distal end of the lower joint element by means of a joint, such as a pivot joint. Particularly preferably, the joint that couples the force transmission element with the lower joint element is located in the knee area of the wearer of the orthopedic device when the orthopedic device is in the mounted state.

For example, the at least one passive actuator is configured to apply a force, especially a tensile force, to the lower joint element and/or the upper joint element. However, since this force is applied eccentrically in relation to the pivot axis about which the two joint elements can be pivoted relative to one another, i.e. the force application point is at a distance from the pivot axis, this force generates a torque whose size depends on the strength of the applied force on the one hand and on the distance between the force application point and the pivot axis on the other.

Preferably, the device is an orthopedic device for supporting a lower back of the wearer. The upper joint element is then preferably an upper body element to be mounted on the wearer’s upper body and the lower joint element is an upper leg element to be mounted on the wearer’s upper leg.

If the user bends over or leans forward when wearing the device, the upper leg element and the upper body element pivot relative to one another. To this end it is advantageous, but not essential, for the upper body element and the upper leg element to be directly connected to one another by a joint, i.e. for one element to be arranged on the respective other element such that it can be pivoted. The torque applied by the at least one passive actuator is then preferably configured in such a way that it supports a pivoting in the opposite direction. As a result, the user of the device is supported when straightening up. Consequently, the torque applied by the at least one actuator is the torque that supports the user of the device when straightening up.

Alternatively, the device is an orthopedic device for supporting a knee joint of the wearer. In this case, the upper joint element is an upper leg element to be mounted on the upper leg and the lower joint element is a lower leg element to be mounted on the wearer’s lower leg.

The orthopedic device has at least one ground contact element, by means of which a force generated from the torque applied by the actuator is transferred to the ground.

Preferably, the at least one ground contact element is arranged in such a way that, when the orthopedic device is in the mounted state, it comes into contact with the ground when the wearer’s foot on which the ground contact element is arranged comes into contact with the ground. The ground contact element is preferably arranged in the heel area on the wearer’s foot, for example on or in a shoe worn by the wearer of the device. This has the advantage that the torque applied by the at least one passive actuator and the force generated as a result can be immediately introduced into the ground, thereby developing the supportive effect.

Alternatively, the ground contact element may also be arranged in such a way that it does not come into contact with the ground when the orthopedic device is in the mounted state as long as no torque is applied to the lower joint element and/or the upper joint element by the passive actuator. This protects the ground contact element because, for example, it does not make contact with the ground with every step taken by the wearer of the device and therefore cannot wear down. In this case, however, the displacement and pivoting of the lower joint element relative to the upper joint element is initially required before a force generated from the torque can be introduced into the ground via the ground contact element.

To be able to introduce a force into the ground, at least one force transmission element is preferably displaced by the force applied by the actuator until the connection that transmits the force is established. As a rule, a compressive force is transmitted to the ground, so that it is sufficient for the connection transmitting the force if the individual components and elements through which the force is transmitted are in contact with each other in such a way that a compressive force can be transmitted. It is often advantageous, but not essential, to fix the components and elements to each other. For example, it is sufficient if the ground contact element comes into contact with the ground and can therefore transmit a compressive force acting on the ground contact element to the ground. It does not need to be fixed to the ground.

The ground contact element is preferably made of a plastic and preferably has a contact surface that is placed against the ground. It preferably has an anti-slip profile. This prevents the ground contact element from slipping relative to the ground. This should be avoided particularly in the case of high loads, i.e. when the user is being supported to a particularly significant extent by the device. The contact surface is preferably curved, for example it is curved in the shape of a circle, so as to guarantee the most uniform and preferably anti-slip design possible, regardless of the orientation in which it comes into contact with the ground.

Preferably, at least one ground contact element is arranged on the force transmission element in such a way that it is displaced along with it. In this case, the force applied by the actuator acts in such a way that the force transmission element on which the ground contact element is located is displaced and the ground contact element is pressed onto the ground. From the moment that the ground contact element comes into contact with the ground, the ground acts as a counter-bearing for the force applied after this moment, which is generated from the torque applied by the at least one actuator, so that the force is diverted into the ground. This occurs almost or completely independently of the orientation and/or position of the ground contact element, so that the effect supporting the user is also achieved when the the user of the device is not standing upright or walking. In a particularly preferred embodiment, the ground contact element is not in contact with the ground when the wearer is standing upright or walking.

Alternatively, a ground contact element can be positioned in such a way that it is in contact with the ground, regardless of whether the wearer of the device is standing upright or walking or, for example, leans forward. Such a ground contact element can be arranged, for example, on the wearer’s shoe or be formed by a shoe. In this embodiment, the at least one force transmission element can be displaced relative to at least one ground contact element. A connection that transmits the force can be established through the displacement of the force transmission element by the force generated from the torque applied by the actuator. The displacement continues until the ground contact element and the force transmission element are in contact with each other in such a way that the connection that transmits the force is established. The force is subsequently transmitted into the ground.

In a preferred embodiment, at least one ground contact element is arranged on a force transmission element in the form of a lower leg element, which is arranged on the lower joint element, in this case an upper leg element, such that it can be pivoted and preferably extends along the wearer’s lower leg. The upper leg element and the lower leg element are preferably connected to each other by a joint with a pivot axis. It is preferably a free-running joint that maps the degree of freedom of the knee.

Preferably, the force transmission element in the form of the lower leg element features a lower leg shell for mounting on the user’s lower leg. Said shell can preferably be displaced along a longitudinal extension of the force transmission element relative to the force transmission element. If the user of the device is standing upright, the upper leg element and the lower leg element arranged thereon extend along the leg direction, i.e. preferably along the direction of the earth’s gravitational field. It has been proven advantageous if, in this state, a combined length of the lower leg element and the upper leg element is insufficient, so the ground contact element arranged on the lower leg element is in contact with the ground. However, if the user of the device kneels down, for example, the distance between the distal end of the upper leg element, which is preferably arranged in the waist or pelvic area of the user, and the ground reduces. The at least one passive actuator applies the torque to the upper leg element, which is pivoted relative to the upper body element until the ground contact element comes into contact with the ground. The ground contact element is preferably arranged on a distal end of the lower leg element.

Of course, the force transmission element can also be designed in the form of a lower leg element, without the ground contact element being located at its distal end. The connection between the ground contact element and the lower leg element is only achieved when the force transmission element, i.e. the lower leg element in this embodiment, is displaced. The lower leg shell preferably remains in its position relative to the lower leg on which it is resting. This is achieved in that the lower leg shell can be displaced along the force transmission element relative to said lower leg. Therefore, when the force transmission element is displaced, for example to establish contact with the ground element or the ground, the lower leg shell does not have to follow this movement, but stays in position on the lower leg. This prevents the lower leg shell from slipping.

Given that the lower leg shell is arranged on the lower leg element such that it can be displaced in the longitudinal direction of the lower leg element, the lower leg element can be displaced in this direction relative to the lower leg shell resting on the lower leg and thus be brought into contact with the ground. In the process, the lower leg shell is preferably not displaced relative to the lower leg. If a force and therefore a torque is now applied to the upper leg element by the at least one passive actuator, the lower leg element and thus also the lower leg element arranged thereon move. Since the user’s foot is preferably on the ground in this state and is not or cannot be displaced relative to the ground, the lower leg element is displaced relative to the lower leg shell due to the movement caused by the at least one passive actuator. As a result, either the force transmission element in the form of the lower leg element comes into contact with the ground contact element or the ground contact element comes into contact with the ground. From this moment, the ground acts as a counter-bearing for the force applied by the actuator.

Preferably, at least one ground contact element is arranged at a distal end of the upper leg element when it forms the lower joint element. This at least one ground contact element is especially advantageous when the user of the devices is kneeling and wants to straighten or stand up. In this state, the at least one knee of the user is on the ground. However, only the toes of the corresponding foot are in contact with the ground. Therefore, a ground contact element at a distal end of a lower leg element cannot come into contact with the ground even by longitudinal displacement of the lower leg element. The at least one ground contact element at the distal end of the upper leg element, on the other hand, comes into contact with the ground as long as the at least one passive actuator pivots the upper leg element relative to the upper body element as a result of the force it applies when the user is in this position. In this embodiment too, as soon as the ground contact element is in contact with the ground, the ground acts as a counter-bearing for the force applied by the at least one passive actuator. In this embodiment, the upper leg element preferably simultaneously acts as a force transmission element.

The at least one ground contact element at the distal end of the upper leg element preferably protrudes frontally over the upper leg element. This applies particularly when the user of the device is standing upright.

Preferably, the force transmission element in the form of the lower leg element or at least a distal section of it can be rotated about a longitudinal axis of the lower leg element. As a result, a freedom of movement of the lower leg and therefore of the foot is not restricted by the device. The lower leg element preferably has a proximal section and a distal section, which are preferably designed as separate components and are both preferably rod or tube-shaped. The two components are preferably connected to each other in such a way that they can be rotated relative to each other about the longitudinal axis of the lower leg element. Here, the longitudinal axis of the lower leg element is not necessarily a symmetrical axis. In particular, it is not necessary, although it is advantageous, for the lower leg element, the proximal and/or the distal section to have a rotational symmetry, the symmetrical axis forming the longitudinal axis. For the embodiments described here to function, it is sufficient if the longitudinal axis of the lower leg element corresponds to the direction of the longitudinal extension.

Preferably, the lower leg shell is arranged at the distal end of the lower leg element.

In a preferred embodiment, the at least one passive actuator has at least one mechanical energy store. This is especially preferably if the device is an orthopedic device for supporting a lower back. In this case, the device preferably features a pelvic element. The upper body element preferably has a first engagement element and the upper leg element has a second engagement element. In this case, the upper leg element is arranged on the pelvic element such that it can be swivelled about a first swivel axis and the upper body element is movably arranged relative to the pelvic element. The first engagement element can be engaged and disengaged with the second engagement element by moving the upper body element relative to the pelvic element and the mechanical energy store can be charged and discharged in that the upper leg element pivots relative to the upper body element when the first engagement element is engaged with the second engagement element.

This embodiment is based on the knowledge that the user’s lower back does not always need support when an angle between the upper body element, which is arranged in the chest or back area of the user’s upper body for example, and the wearer’s upper leg falls below a predetermined angle, i.e. the two body parts are pivoted against each other. Rather, support is only required when a pivoting occurs between the wearer’s upper body, for example in the chest, and the wearer’s pelvis. The device ensures that a supporting force is always exerted when this pivoting between the wearer’s upper body and pelvis occurs. If, on the other hand, the upper body is pivoted relative to the upper leg, without the upper body moving relative to the pelvis, no force should be applied, where applicable.

The first engagement element and the second engagement element are designed in such a way that a force can be transmitted between the two when they are engaged and no force is transmitted when they are not engaged. In addition, they are designed in such a way that they can be engaged and disengaged multiple times. They are preferably two form-fitting elements and/or two force-locking elements, especially preferably two frictional elements.

In this embodiment, the upper body element, which is preferably arranged on the wearer’s back or chest, has to move relative to the pelvic element, which is preferably arranged on the wearer’s pelvis, in order to engage the first engagement element with the second engagement element. Only then can the energy store of the at least one passive actuator be charged with mechanical energy or discharged by further pivoting. Without this movement of the upper body element relative to the pelvic element, the two engagement elements remain disengaged and a movement of the upper leg element relative to the upper body element does not result in the energy store being charged with energy. Therefore, a force that supports the extension cannot be applied.

Preferably, the mechanical energy store comprises at least one spring element or is a spring element. Alternatively or additionally, it has at least one accumulator, a pneumatic and/or a hydraulic system and/or a hydraulic energy store.

This embodiment of the orthopedic device consequently ensures that no additional force supporting the extension is applied when sitting or climbing stairs, in which case there is usually insufficient movement between the upper body element and the pelvic element, whereas support is achieved when lifting heavy objects, for example.

With this type of movement, the upper body element is moved relative to the pelvic element and the first engagement element engaged with the second engagement element. If, in this state, the upper leg element is pivoted relative to the pelvic element, the mechanical energy store is charged with potential energy, causing a force supporting the extension to be applied.

In a preferred embodiment, the first engagement element features a gearwheel, which is arranged eccentrically on the pelvic element such that it can be pivoted about a second pivot axis. In this embodiment, the second engagement element is preferably a gearwheel that is arranged on the upper leg element such that it is torque-proof.

Preferably, the upper body element is connected with the first engagement element and particularly with the gearwheel of the first engagement element in such a way that the first engagement element is pivoted about the second pivot axis when the upper body element is moved relative to the pelvic element. This movement causes the gearwheel of the first engagement element to come into contact with the second engagement element, which preferably also features a gearwheel, and to be engaged with it. If, in this state, the upper leg is moved relative to the upper body, for example by the wearer kneeling down, the mechanical energy store is charged with potential energy.

During the opposite movement, this potential energy is initially released in that a force is exerted on the upper leg element and/or the upper body element which supports the extension of the wearer’s body. In the process, the upper leg element is first pivoted relative to the upper body element until at least one ground contact element comes into contact with the ground, which then acts as a counter-bearing. Only when the upper body element is moved back relative to the pelvis and thus relative to the pelvic element fixed to the pelvis is the first engagement element disengaged from the second engagement element and the mechanical energy store cannot be recharged or discharged further.

Advantageously, the upper body element and the first engagement element have connecting elements that correspond to each other, so that the upper body element can be connected to the first engagement element in multiple positions. For example, the upper body element may comprise a projection or a peg or pin, which can be inserted into recesses or depressions on the first engagement element. Of course, the reverse embodiment is also possible, in which the first engagement element features a projection, peg or pin and the recesses and depression are located on the upper body element. Regardless of the actual embodiment, it is advantageous if the upper body element and the first engagement element can be fixed in different positions and relative orientations to each other. Particularly preferably, these different positions mean that the first engagement element is positioned in different angular positions about the second swivel axis. This allows adjustment of how much the upper body element must be moved relative to the pelvic element to engage the two engagement elements. Consequently, it allows adjustment of the point in time during a movement at which the mechanical energy store can be charged.

Particularly preferably, the orthopedic device features a displacement device that is configured to move the first engagement element and/or the second engagement element towards each other when the upper body assumes an angle relative to the pelvic element that is smaller than a predetermined limit angle. The angle between the upper body element and the pelvic element is approximately 180° when the wearer of the device is standing upright. If they bend over or tilt the upper body relative to the pelvic element, this angle becomes smaller. If the angle passes the predetermined limit angle in the process, the angle is then smaller than this predetermined limit angle, so that the displacement device moves the two engagement elements towards each other. Preferably, the displacement device moves either the first engagement element or the second engagement element, while the respective other engagement element remains stationary. Alternatively, the displacement device moves both the first engagement element and the second engagement element.

In contrast to the embodiment described further above, in which the two engagement elements are moved continuously towards each other when the upper body element is moved relative to the pelvic element and are engaged when the limit angle is reached, in the embodiment described here no movement of the two force transmission elements towards each other is initially achieved. Only when the angle between the upper body element and the pelvic element passes the predetermined limit angle does the movement described here take place, so that the two engagement elements are engaged with each other afterwards.

Particularly preferably the displacement device is configured to move the first engagement element and/or the second engagement element away from each other when the upper body assumes an angle relative to the pelvic element that is larger than a predetermined limit angle.

In order to move the first engagement element and/or the second engagement element, the displacement device is configured to exert a force on the respective engagement element to be moved. It has been proven advantageous if this force is maintained after the respective engagement element has been moved and the two engagement elements have been engaged or disengaged from one another. This prevents an inadvertent change in state. If the two engagement elements have been engaged due to a force of the displacement device, the force used to achieve this remains intact so as to prevent the two engagement elements from being inadvertently disengaged, which would affect the functionality of the orthopedic device. The same applies for the force that disengages the two engagement elements. This force also remains intact so as to prevent an inadvertent displacement of the respective engagement element, which would cause the two engagement elements to re-engage with each other.

Preferably, at least two magnets are arranged on the pelvic element or the upper leg element and at least one magnet is arranged on the respective other element in such a way that they exert a force on each other, the direction of which changes when the angle passes the predetermined limit angle during movement of the upper body element relative to the pelvic element. In this embodiment, the displacement device consequently features the magnets described. On the element on the upper body element or on the pelvic element on which at least two magnets are arranged, these are preferably arranged in different orientations. This means that the north pole of at least one of the magnets and the south pole of at least one other magnet are directed towards the respective other element of the orthopedic device.

If the angle between the upper body element and the pelvic element is greater than the predetermined limit angle, the two engagement elements are not engaged with each other. Preferably, a force is consequently exerted that keeps the two engagement elements away from each other. This can be achieved by the magnets exerting a force on one another. This may be a repulsive force, for example. This is achieved by a magnet of the pelvic element and a magnet of the upper leg element being positioned close to each other, so that their like poles, i.e. south pole or north pole respectively, are directed towards each other. If the pelvic element is now moved relative to the upper leg element, the magnets arranged on the respective elements are also moved. This results in a displacement towards each other of the magnets moved. At the moment at which the angle of the upper body element relative to the pelvic element passes the predetermined limit angle, a second magnet of the pelvic element or the upper leg element preferably enters the area of the at least one magnet of the respective other element. This results in an attracting force, since unlike poles of the two magnets are directed towards each other.

Advantageously, the orthopedic device can be brought into an active and a passive state. So far, the active state has been described and is characterized in that the first engagement element and the second engagement element can be engaged and disengaged from one another by moving the upper body element relative to the pelvic element. This is not possible in the passive state. In the passive state, if the upper body element is moved relative to the pelvic element, the two engagement elements do not engage or disengage from one another.

Preferably, the device has at least one actuating element, the actuation of which allows the device to be moved from the active state into the passive state and/or vice-versa. With such an actuating element, a movement of the first engagement element can be decoupled from the movement of the upper body element, for example. By actuating the actuating element again, the movement is coupled again so that the engagement elements can be engaged.

### DESCRIPTION OF THE DRAWINGS

In the following, an embodiment example of the invention will be explained in more detail with the aid of the accompanying figures.

FIG. 1 depicts a device according to an embodiment example of the present invention in the mounted state.

FIG. 2 depicts the device of FIG. 1 in a different movement situation than is shown in FIG. 1.

FIG. 3 depicts yet another movement situation with the device of FIG. 1.

FIG. 4 depicts a further embodiment example of the present invention and in which the user wearing the orthopedic device is kneeling.

FIG. 5 corresponds to the device of FIG. 1, and shows an embodiment designed in such a way that it is in contact with the ground as long as the user’s foot is in contact with the ground, regardless of the position of the user.

FIG. 6 depicts a similar representation of the device shown in FIG. 5 with the same ground contact element, but where the upper leg element is longer.

FIG. 7 depicts a device similar to that shown in FIG. 1, but depict an embodiment where an active actuator is included.

FIG. 8 depicts a device similar to that shown in Figure, but depicts an embodiment with a spring as the actuator.

### DETAILED DESCRIPTION

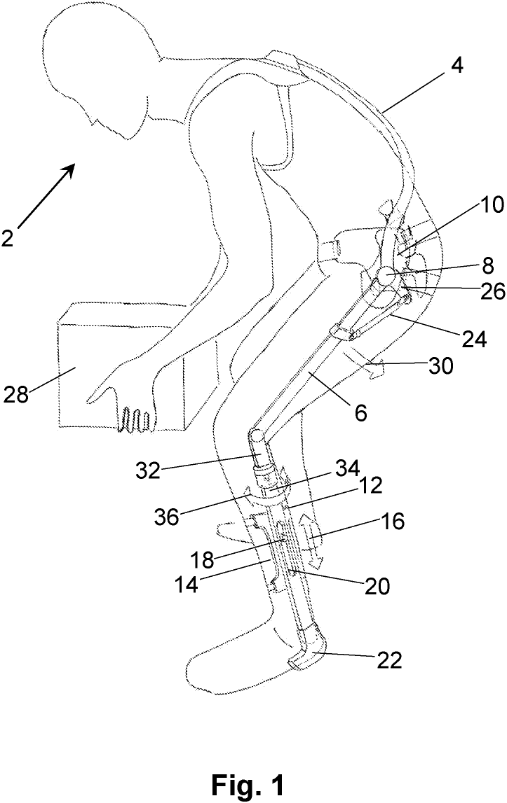

FIG. 1 depicts a device according to an embodiment example of the present invention in the mounted state. A user 2 is wearing the device, which comprises an upper body element 4 arranged on an upper body of the user 2 and an upper leg element 6. The upper leg element 6 is arranged on a pelvic element 10 such that it can be pivoted about a pivot axis 8 and can thus also be pivoted relative to the upper body element 4, which is also movably arranged at the distal end of the pelvic element 10. A force transmission element in the form of a lower leg element 12 is arranged at the distal end of the upper leg element 6, a lower leg shell 14 being arranged on said lower leg element. Th lower leg shell 14 is attached to a lower leg of the user 2.

The lower leg shell 14 is arranged such that it can be displaced longitudinally relative to the lower leg element 12, as indicated by the double arrow 16. In the embodiment example shown, the lower leg shell 14 features a projection 18 in the form of a peg, which is slidably mounted in an elongated hole 20 arranged for this purpose on the lower leg element. At the distal end of this lower leg element is a ground contact element 22, which is in contact with the ground in the figure depicted in FIG. 1.

The device also comprises an actuator 24 which, in the embodiment example shown is a passive actuator, is arranged between the upper leg element 6 and a force application lever 26 of the upper body element 4. In the situation shown in FIG. 1, the user 2 has leaned forward to lift an object 28. Their upper body with the upper body element 4 located thereon has been pivoted relative to the upper leg element 6. The actuator 24, which may be a spring element such as an expander, was tensioned in the process and now exerts a force that pulls the upper leg element 6 in the direction of the arrow 30. As a result, a torque is applied to the upper leg element 6 about the pivot axis 8 in the anti-clockwise direction.

The upper leg element 6 initially follows this torque and is pivoted about the pivot axis 8 in the specified direction. In the process, the force transmission element in the form of the lower leg element 12 located at the distal end of the upper leg element 6 is also moved and, in FIG. 1, displaced downwards until the situation shown in FIG. 1 is achieved and the ground contact element 22 touches the ground. The lower leg element 12 is pivoted relative to the lower leg shell 14. As soon as the ground contact element 22 touches the ground, a further movement of the lower leg element 12 in this direction is no longer possible and the ground acts as a counter-bearing for the force applied by the actuator 24, said force now supporting the user 2 when straightening up.

In the embodiment example shown, the lower leg element 12 has a proximal section 32 and a distal section 34, which can be twisted relative to each other along the double arrow 36.

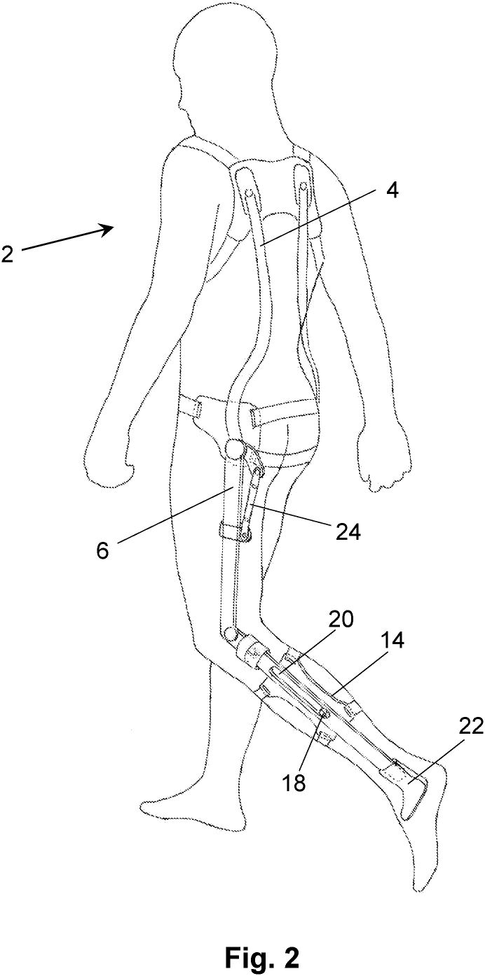

FIG. 2 shows the device from FIG. 1 in another movement situation of the user 2. In the situation depicted, the upper leg element 6 is not pivoted relative to the upper body element, so that the actuator 24 was not tensioned and therefore no force was exerted. The ground contact element 22 therefore does not protrude beyond the sole of the foot of the user 2 and therefore does not represent a restriction of movement. It is clear that the projection 18 is positioned in the elongated hole 20 in a significantly distally displaced manner compared to the situation in FIG. 1. This means that the lower leg element 12 is displaced further proximally, i.e. towards the knee, relative to the lower leg shell 14 compared to the situation in FIG. 1.

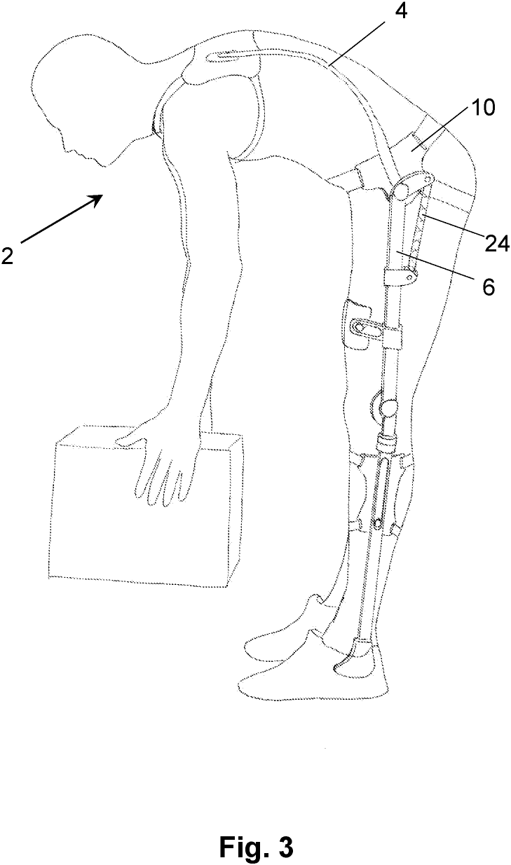

FIG. 3 depicts another situation. The user 2 is leaning their upper body forward with extended legs. The upper body element 4 features a first engagement element, not depicted, and the upper leg element 6 has a second engagement element, also not depicted. In the situation shown in FIG. 3, these elements are not engaged with one another, so that the actuator 24 does not exert a force, even though the upper body element 4 is pivoted relative to the upper leg element 6.

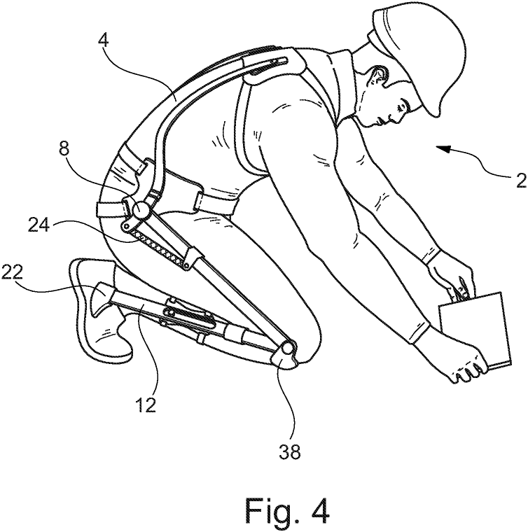

FIG. 4 depicts a kneeling user 2 wearing an orthopedic device according to a further embodiment example of the present invention. It comprises the upper body element 4 and the upper leg element 6, between which the actuator 24 is arranged. When the user 2 is in this position, the ground contact element 22 at the distal end of the lower leg element 12 is not arranged in a way that allows it to be brought into contact with the ground by the torque applied by the actuator 24. The device has another ground contact element 38 for this case. It is preferably arranged at a distal end of the upper leg element 6, which acts as a force transmission element. As a result of the torque applied by the actuator 24, the upper leg element 6 is pivoted about the pivot axis 8 in the clockwise direction until the ground contact element 38 comes into contact with the ground. The ground then acts as a counter-bearing for the force applied by the actuator 24.

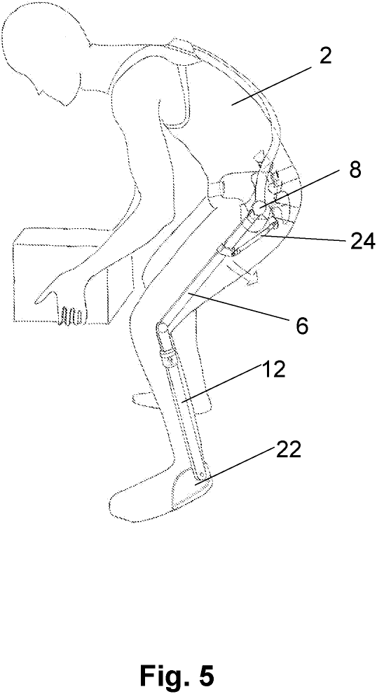

FIG. 5 largely corresponds to the representation in FIG. 1, the ground contact element 22 in FIG. 5 being designed in such a way that it is in contact with the ground as long as the user’s foot is in contact with the ground, regardless of the position of the user 2. A displacement device, as it is shown in FIG. 1 and which enables the displacement along the double arrow 16 in FIG. 1, is not necessary in the embodiment in FIG. 5. The upper leg element 6, which forms the lower joint element, is articulated with the lower leg element 12, which forms the force transmission element. The actuator 24 exerts a torque in that a tensile force is applied away from the pivot axis 8, i.e. eccentrically in relation to this axis, by the actuator 24.

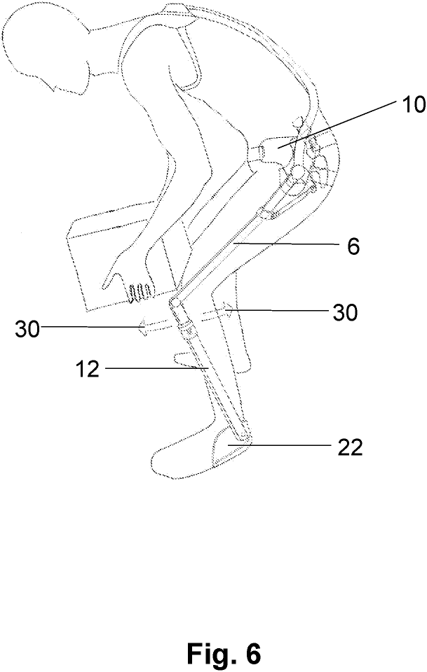

FIG. 6 depicts the representation from FIG. 5 with the same ground contact element 22. It is different to FIG. 5 in that the upper leg element 6, i.e. the lower joint element, is longer. There is no connection of the upper leg element 6 or the lower leg element 12 to the wearer’s leg. This results in freedom of movement along the arrows 30. The joints, which connect the pelvic element 10 to the upper leg element 6, the lower leg element 12 and the ground contact element 22, only enable a movement in one plane and breaking out of this plane is not possible.

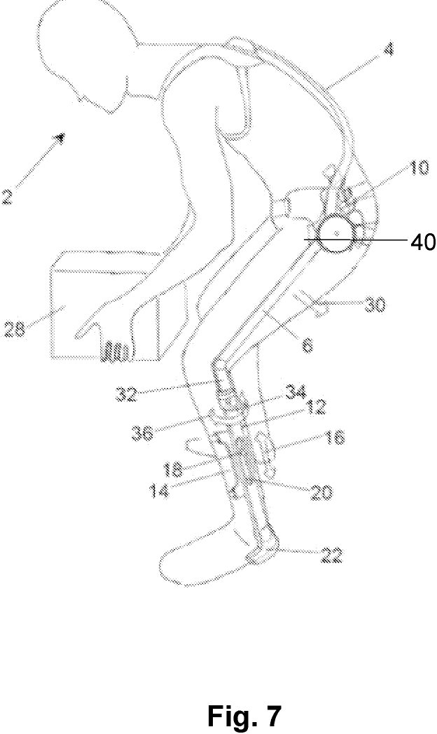

FIG. 7 shows the representation from FIG. 1 with the difference that instead of the exemplary passive actuator shown in FIG. 1, an active actuator 40 is present. This is designed as a motor and is set up to apply the necessary torque to move the upper leg element 6 relative to the pelvic element 10 and/or to the upper body element 4. The active actuator 40 is preferably an electric motor. This has the advantage of being able to be designed to be quiet and small. As an energy source, the device according to FIG. 7 has an energy storage unit that is not shown, which is, for example, a rechargeable battery and in which electrical energy can be stored.

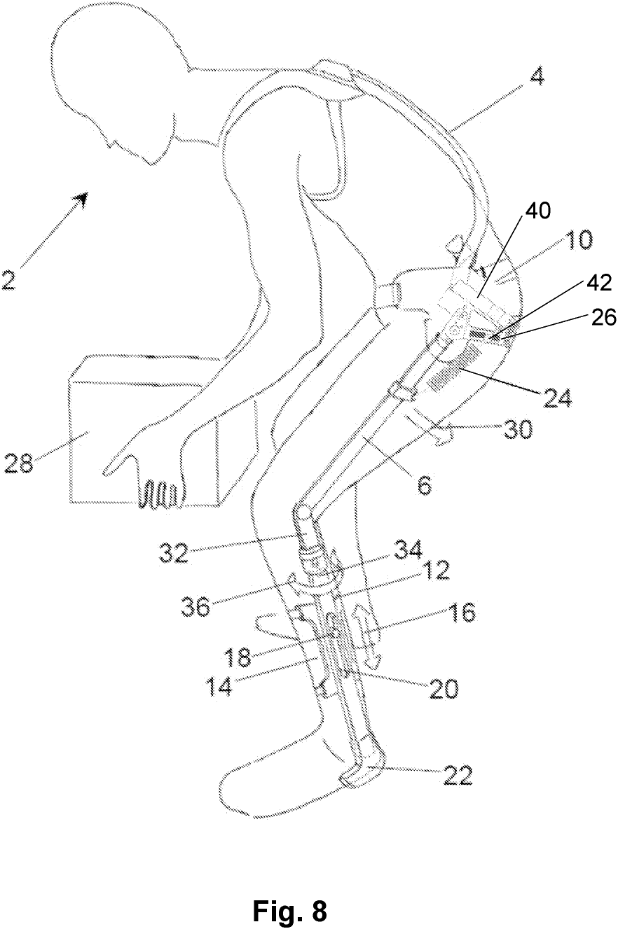

FIG. 8 shows another embodiment which has a passive actuator 24 in the form of a spring. This engages the force application lever 26 and thus exerts the force and, from this, the necessary torque. A force application point 42, at which the passive actuator 24 engages the force application lever 26, is formed to be displaceable along the force application lever 26. To displace the force application point 42, an electric motor 44 is actuated. However, the passive actuator 24 itself is not connected to its own drive or to any power source other than the user 2.

Reference list:

| | |
|---|---|
| 2 | user |
| 4 | upper body element |
| 6 | upper leg element |
| 8 | pivot axis |
| 10 | pelvic element |
| 12 | lower leg element |
| 14 | lower leg shell |
| 16 | double arrow |
| 18 | projection |
| 20 | elongated hole |
| 22 | ground contact element |
| 24 | passive actuator |
| 26 | force application lever |
| 28 | object |
| 30 | arrow |
| 32 | proximal section |
| 34 | distal section |
| 36 | double arrow |
| 38 | ground contact element |

1. An orthopedic device for supporting a lower limb and/or a lower back of a user, comprising:
- at least one upper joint element,
- at least one lower joint element, wherein the at least one upper joint element and the at least one lower joint element are pivotablerelative to each other,
- at least one actuator configured to apply a torque to the at least one upper joint element and/or the at least one lower joint element for pivoting the at least one upper joint element and the at least one lower joint element relative to each other, and
at least one ground contact element connected to the at least one lower joint element such that when the orthopedic device is in a mounted state, a force is transmitted by the at least one ground contact element to a ground on which the user is located as a result of torque applied by the at least one actuator.

2. The orthopedic device according to claim 1, wherein the at least one ground contact element is connected to the at least one lower joint element via at least one force transmission element.

3. The orthopedic device according to claim 2, wherein the at least one ground contact element is arranged on the at least one force transmission element such that the at least one ground contact element is displaced with the at least one force transmission element.

4. The orthopedic device according to claim 2, wherein the at least one force transmission element is displaceable relative to the at least one ground contact element and wherein a connection that transmits force is established between the at least one force transmission element and the at least one ground contact element by the at least one force transmission element being displaced by the torque applied by the at least one actuator.

5. The orthopedic device according to claim 2, wherein the at least one force transmission element is pivotably arranged on the at least one lower joint element.

6. The orthopedic device according to claim 5, wherein the at least one force transmission element comprises a lower leg shell mountable on a lower leg of the user that is along a longitudinal extension of the at least one force transmission element relative to the at least one force transmission element.

7. The orthopedic device according to claim 1 wherein the at least one ground contact element is arranged at a distal end of the at least one lower joint element (.

8. The orthopedic device according to claim 7 wherein the at least one ground contact element protrudes frontally over the at least one upper joint element.

9. The orthopedic device according to claim 1 wherein the at least one force transmission element or a distal section of the at least one force transmission element is rotatable about a longitudinal axis of the at least one force transmission element.

10. The orthopedic device according to claim 9 wherein a 8 lower leg shell is arranged on the distal section of the at least one force transmission element.

11. The orthopedic device according to claim 1 wherein the at least one actuator comprises a mechanical energy store.

12. The orthopedic device according to claim 10, wherein the at least one upper joint element is an upper body element and the at least one lower joint element is an upper leg element, and wherein the orthopedic device further comprises a pelvic element, wherein the upper body element comprises a first engagement element and the upper leg element comprises a second engagement element, wherein
- the upper leg element is arranged on the pelvic element such that the upper leg element is pivotatableabout a first pivot axis ,
- the upper body element is movably arranged relative to the pelvic element,
- the first engagement element is engageable and/or disengageable from the second engagement element by moving the upper body element relative to the pelvic element, and
- the mechanical energy store is chargeable and/or dischargeable by pivoting the upper leg element relative to the upper body element when the first engagement element is engaged with the second engagement element.
